(19) United States
(12) Patent Application Publication
(10) Pub. No.: US 2012/0058099 A1
(43) Pub. Date: Mar. 8, 2012

(54) USE OF HYPOXANTHINE FOR PROMOTION OF NEURONAL OUTGROWTH

(75) Inventor: Carleen Ann Irwin, Chestnut Hill, MA (US)

(73) Assignee: CHILDREN'S MEDICAL CENTER CORPORATION, Boston, MA (US)

(21) Appl. No.: 13/254,804

(22) PCT Filed: Sep. 10, 2009
(86) PCT No.: PCT/US09/56566

§ 371 Date: Nov. 21, 2011

Related U.S. Application Data
(60) Provisional application No. 61/157,487, filed on Mar. 4, 2009.

(51) Int. Cl. A61K 31/522; A61K 38/18; A61K 38/44; A61K 31/708; A61K 38/17; C12N 5/0793; A61P 25/28; A61P 27/06; A61P 25/14; A61P 25/16; A61P 25/08; A61K 31/7004; A61P 25/00

(52) U.S. Cl. 424/94.4; 514/263.3; 514/23; 514/8.9; 514/8.4; 514/45; 514/17.7; 514/17.9; 514/17.8; 514/20.8; 435/375

(57) ABSTRACT

Disclosed herein is a method of promoting neuronal outgrowth in a neuron. The method comprises contacting the neuron with an effective amount of hypoxanthine, to thereby promote neuronal outgrowth of the neuron. The hypoxanthine may be contacted in the absence of xanthine oxidase and/or in the absence of exogenous nerve growth factor (NGF), and/or in the absence of exogenous D-mannose, and/or in the absence of exogenous oncomodulin, and/or in the absence of exogenous TGF-B. The neuron may be an optic nerve neuron or a retinal neuron and/or an injured neuron. Neurons may be from the central nervous system (CNS) or the peripheral nervous system (PNS). The methods are useful for treating an injured neuron, for example to an optic nerve neuron, resulting from branch and central vein/artery occlusion, trauma, edema, angle-closure glaucoma, open-angle glaucoma. Methods of treatment of various neurological injuries and diseases, as well as therapeutic compositions, are also disclosed.

### CROSS REFERENCE TO RELATED APPLICATIONS

This application claims the benefit under 35 U.S.C. 119(e) of U.S. Provisional Patent Application Serial No. 61/157,487, filed Mar. 04, 2009, the contents of which is herein incorporated by reference in their entirety.

### GOVERNMENT SUPPORT

This invention was supported by the National Institutes for Health (NIH) Grant No. EY05690 and the Government of the United States has certain rights thereto.

### FIELD OF THE INVENTION

The present invention relates to the induction of neurite outgrowth by exogenously added hypoxanthine, and treatment of conditions which will benefit from such neurite outgrowth by administration of pharmaceutical compositions containing exogenous hypoxanthine.

### BACKGROUND

Hypoxanthine is a purine base formed as an intermediate in the degradation of purines and purine nucleosides to uric acid and in the salvage of free purines. It is present in muscle and other tissues, formed during uric acid synthesis. When hypoxanthine is complexed with ribose it is inosine. Inosine has been found to promote neuronal outgrowth in mammalian neurons. Evidence indicates that there may be little conversion of inosine to hypoxanthine in neurons; adenosine produced by neurons is metabolized to hypoxanthine by astrocytes (J. Neurosci Res. 86(15): 3447-3455 (2008)). PNP, the enzyme that converts inosine to hypoxanthine is not detectable in neurons by immunohistochemistry, but is seen in astrocytes and microglia (Castellano et al., J. Histochem. Cytochem. 38: 1535-1539 (1990)). There is little PNP activity in neurons, compared to glia (Ceballo et al., J. Neurochem. 62: 1144-1153 (1994)).

### DESCRIPTION OF THE FIGURES

FIG. 1 contains two separate sets of bar graphs and line graphs representative of data obtained from embryonic cortical neuron competition experiments. FIG. 1A displays data in the form of a bar graph representative of neuronal outgrowth resulting from exposure to the indicated amounts of inosine, in the presence of 0 μm, 5 μm, 10 μm, 20 μm, 40 μm, 80 μm of the inhibitor 6-thioguanine. FIG. 1B displays data similarly displayed of neuron axonal outgrowth resulting from exposure to the indicated amounts of hypoxanthine, in the presence of 0 μm, 5 μm, 10 μm, 20 μm, 40 μm, 80 μm of the 6-thioguanine. 6-thioguanine competitively inhibits the actions of both inosine and hypoxanthine.

FIG. 2 is a bar graph representative of neuronal outgrowth resulting from exposure to BDNF, inosine, and hypoxanthine. Both inosine and hypoxanthine were also given in the presence of the inhibitor BCX-34 (BCX). BCX-34 blocks the conversion of inosine to hypoxanthine.

FIG. 3 is a series of photographs of embryonic cortical neurons grown in the presence of the indicated factors (BDNF, hypoxanthine, BDNF+6-thioguanine, BDNF+6-thioguanine+hypoxanthine), which have then been stained with either antibodies to Taul, antibodies to MAP2, or both antibodies to Taul and antibodies to MAP2, in order to visualize neuronal outgrowth. BDNF and hypoxanthine cause outgrowth. BDNF stimulated outgrowth can be inhibited by 6-TG and this inhibition can be overcome by adding high amounts of hypoxanthine.

### DETAILED DESCRIPTION

The present invention relates to the use of hypoxanthine in methods for promoting neuronal outgrowth. The invention is based, at least in part, on the discovery that hypoxanthine induces outgrowth of neurons. As such, aspects of the present invention relate to methods of inducing/promoting neuronal outgrowth by contacting hypoxanthine to a neuron.

As used herein, the language “hypoxanthine” is art recognized. The term hypoxanthine, as used herein, refers to exogenously added or administered hypoxanthine. The exogenous hypoxanthine used in the methods described herein has been isolated and/or purified away from its natural state in a cell or a body, or a bodily fluid, secretion, or extract. In one embodiment, the isolated and/or purified hypoxanthine is present in a formulation (e.g., pharmaceutical composition) with other components, described herein. The term hypoxanthine, or administering or contacting hypoxanthine, is not intended to encompass hypoxanthine that results from a breakdown product of another exogenously added molecule (e.g. exogenous or endogenous inosine). The term “exogenous” is used to represent an external composition that is provided, administered or contacted to an organisms from an external source. This is opposed to the term “endogenous” which is used to represent a composition that is naturally present within an organisms (e.g. cell, whole body, bodily fluid, secretion or extract). The methods described herein are also expected to further encompass the use of analogs of hypoxanthine which retain the neuron outgrowth activity of hypoxanthine, such as AIT-082 (Geerts, ID rugs 1(6):694-9 (1998)) and guanine. The term “hypoxanthine analog” excludes inosine, adenosine, adenine, etc.

One aspect of the invention relates to a method of promoting neuronal outgrowth in a neuron by contacting the neuron with an effective amount of exogenous hypoxanthine. In one embodiment, the hypoxanthine is contacted to the neuron in the presence of one or more exogenous factors (e.g., NGF, BDNF, a hexose or hexose derivative, oncomodulin, TGF-β, inosine, other axogenic factors) described herein. In another embodiment, the hypoxanthine is contacted to the neuron in the absence of one or more exogenous factors described herein (e.g., NGF, BDNF, a hexose or hexose derivative, oncomodulin, TGF-β, inosine, other axogenic factors). In one embodiment, contacting occurs following neuronal injury, not prior to, or concurrent with injury.

Evidence indicates that there is little conversion of inosine to hypoxanthine in neurons (J. Neurosci Res 86(15): 3447-3455 (2008)). As such, administration of hypoxanthine rather than inosine to neurons is expected to provide superior therapeutic benefit since it would work directly on the neurons, whereas the inosine would require conversion by the surrounding glial cells. Superior therapeutic benefit is further expected for conditions where surrounding glial cells have been reduced or eliminated, e.g., from injury or disease.

The contacting of the hypoxanthine to the neuron can occur in vivo or in vitro. The neuron is contacted with the hypoxanthine under conditions appropriate for growth and/or maintenance of the neuron (e.g. the environment of the neuron provides the appropriate nutrients, temperature, etc.). An effective amount is an amount which delivers sufficient hypoxanthine to the target neuron(s) to produce a detectable, amount of neuronal outgrowth. Detection can be by physical examination of the neuron(s) or by functional analysis as described herein. Contacting in vitro is often by including the agent in the media in which the cells or tissue are grown. Contacting in vivo is generally achieved by administration of hypoxanthine to a subject in which the target neuron(s) resides. One of skill in the art will recognize that the effective amount for in vivo contact may require a higher dose of administration to result in a sufficient amount of hypoxanthine reaching the target neuron.

As the term is used herein, a “target neuron” is a neuron in which outgrowth is desired. The target neuron is a neuron which the hypoxanthine is directed toward ultimately contacting, to thereby produce outgrowth.

As used herein, the term “neuronal outgrowth” refers to the process by which axons and/or dendrites grow out of a neuron. The outgrowth can result in a totally new axon, increased growth of an already extended axon, or the repair of a partially damaged axon. Outgrowth is typically evidenced by extension of an axonal process of at least 5 cell diameters in length.

As used herein, the language “inducing the outgrowth of neurons” is intended to include the capacity to stimulate outgrowth of neurons to various levels, e.g., to levels which allow for the treatment of targeted neuronal injuries.

### Types of Neurons

In one embodiment the neurons are mammalian neurons, for example, neurons from a primate (e.g. human neurons). Other examples of mammalian neurons include those from animals typically kept as pets (e.g., dogs, cats, birds, horses), farm animals (e.g., goats, pigs, cows, horses, sheep, poultry fowl) and exotic animals or zoo animals. In one embodiment, the neuron is not a rabbit neuron.

In one embodiment, one or more central nervous system (CNS) neurons are contacted with hypoxanthine. In one embodiment, the CNS neuron is unresponsive to nerve growth factor (NGF). The term CNS neuron is not intended to include support or protection cells such as astrocytes, oligodentrocytes, microglia, ependyma and the like, nor is it intended to include peripheral nervous system (e.g., somatic, autonomic, sympathetic or parasympathetic nervous system) neurons. Although, in some embodiments, such cells are also contacted with hypoxanthine.

In another embodiment, one or more peripheral nervous system neurons are contacted with hypoxanthine. As used herein, the term “PNS (peripheral nervous system) neurons” is intended to include the neurons commonly understood as categorized in the peripheral nervous system, including sensory neurons and motor neurons. The term PNS neuron is not intended to include support or protection cells such as astrocytes, oligodentrocytes, microglia, ependyma and the like, nor is it intended to include CNS nervous system neurons. Although, in some embodiments, such cells are also contacted with hypoxanthine.

In one embodiment, the neuron which is contacted is injured prior to the contact (herein referred to as an “injured neuron”).

In one embodiment, the neuron is an optic nerve neuron or a retinal neuron.

### Treatment Methods

Another aspect of the invention relates to method for treatment/treating a subject with an injured neuron, by administration of hypoxanthine to the subject with the injured neuron. Administration is performed to promote contact of an effective amount of the administered hypoxanthine to the injured neuron within the subject. A therapeutically effective amount of the hypoxanthine or pharmaceutical composition containing hypoxanthine is administered to the subject. The method may further comprise selecting a subject in need of such treatment (e.g., identification of a damaged nerve, (e.g., CNS and/or PNS). The injury may occur by one or more means described herein. In one embodiment, administration occurs following neuronal injury in the subject, not prior to, or concurrent with injury.

As used herein, the term “subject” is intended to include animals susceptible to neuronal (e.g., CNS or PNS) injuries, mammals, such as primates (e.g. human). Other examples of subjects include animals typically kept as pets (e.g., dogs, cats, birds, horses), farm animals (e.g., goats, pigs, cows, horses, sheep, poultry fowl) and exotic animals or zoo animals. In one embodiment, the subject is not a rabbit.

Another aspect of the invention relates to method for treatment/treating a disease, disorder or condition associated with damage to a neuron or reduced neuronal outgrowth of a neuron, by administration of hypoxanthine to a subject with such a disease, disorder or condition. As used herein, the term “associated with” injury to a neuron, when used in conjunction with disease, disorder or condition, refers to the damage or injury to the neuron resulting from or causing the disease symptoms. Such disorders or conditions are generally known in the art, with representative diseases, disorders and conditions so associated being described herein. The subject may be at risk for such a disease, disorder or conditions or suspected of being in the early stages of development. Prevention of the development or progression of such a disease, disorder or condition by the methods described herein is also envisioned. For treatment or prevention, a therapeutically effective amount of the hypoxanthine is administered. Hypoxanthine can be administered in the presence of one or more exogenous factors (e.g., NGF, BDNF, a hexose or hexose derivative, oncomodulin, TGF-β, inosine, other axogenic factors, a cAMP activator) described herein. For instance, they can be co-administered, or administered separately. In another embodiment, the hypoxanthine is administered in the absence of one or more exogenous factors described herein (e.g., NGF, BDNF, a hexose or hexose derivative, oncomodulin, TGF-β, inosine, other axogenic factors, a cAMP activator).

The method may further comprise selecting a subject in need of treatment or prevention of nerve damage (CNS and/or PNS). Such selection may involve identification within a subject of nerve damage and/or identification of a risk for the development of nerve damage in the subject.

The methods described herein may further comprise the step of detecting a resultant neuronal outgrowth following treatment or in the course of treatment. For in vitro applications, the therapeutic effect of neural regeneration can be detected by any routinely used method such as a neurite outgrowth assay. For in vivo applications, neuronal outgrowth can be detected by detecting neural regeneration. Such regeneration can be detected using imaging methodologies such as MRI. More commonly, neural regeneration will be detected inferentially by neurological examination showing improvement in the patient's neural function. The detecting step may occur at any time point after initiation of hypoxanthine administration, e.g., at least one day, one week, one month, three months, six months, after initiation of treatment. In certain embodiments, the detecting step will comprise an initial neurological examination and a subsequent neurological examination conducted at least one day, week, or month after the initial exam. Improved neurological function at the subsequent exam compared to the initial exam indicates resultant neural regeneration. The specific detection and/or examination methods used will usually be based on the prevailing standard of medical care for the particular type of neural damage being evaluated (i.e. trauma, neurodegeneration, etc.).

Administration of hypoxanthine to a subject, alone or in combinations described herein is to be made under conditions effective to stimulate nerve regeneration at the site of the injury and/or under conditions effective to at least partially restore nerve function (e.g., through an injured spinal cord). Restoration of nerve function can be evidenced by restoration of nerve impulse conduction, a detectable increase in conduction action potentials, observation of anatomical continuity, restoration of more than one spinal root level, an increase in behavior or sensitivity, or a combination thereof. Administration is by a method which results in contacting the administered factors with the site of injury to thereby promote nerve regeneration (complete or partial).

### Injuries, Diseases and Disorders

The injury of the neuron for treatment can be acute or chronic. An injury can be complete severing, or partial severing of the neuron, or crushing or compression injury to the neuron. In one embodiment, an injury to a neuron directly impairs the normal functioning of the neuron. In another embodiment, the injury to the neuron indirectly impairs the normal functioning of the neuron. The injury to a neuron can result from an acute or traumatic event, chronic event, pressure build-up, chronic neurodegeneration. Injury to a subject often results in injury to a neuron. Causes of neuronal injury include, without limitation, disease and/or infection, ischemia, anoxia, hypoglycemia, contusion, laceration, trauma to the brain or spinal cord (such as caused by acute spinal cord damage or stroke), damage by exogenous chemical agents (e.g., exposure to a toxin), and combinations thereof.

As used herein, the term “stroke” is art recognized and is intended to include sudden diminution or loss of consciousness, sensation, and voluntary motion caused by rapture or obstruction (e.g. by a blood clot) of an artery of the brain. A spinal cord injury may be a complete severing of the spinal cord, a partial severing of the spinal cord, or a crushing or compression injury of the spinal cord.

The neuronal injury may also be associated with peripheral neuropathies including, but not limited to, the following: diabetic neuropathies, virus-associated neuropathies, including acquired immunodeficiency syndrome (AIDS) related neuropathy, infectious mononucleosis with polyneuritis, viral hepatitis with polyneuritis; Guillian-Barre syndrome; botulism-related neuropathy; toxic polyneuropathies including lead and alcohol-related neuropathies; nutritional neuropathies including subacute combined degeneration; angiopathic neuropathies including neuropathies associated with systemic lupus erythematosis; sarcoid-associated neuropathy; carcinomatous neuropathy; compression neuropathy (e.g. carpal tunnel syndrome) and hereditary neuropathies, such as Charcot-Marie-Tooth disease, peripheral nerve damage associated with spinal cord injury can also be treated with the present method.

Peripheral nerves such as dorsal root ganglia, otherwise known as spinal ganglia, are known to extend down the spinal column. These nerves can be injured as a result of spinal injury. Such peripheral nerve damage associated with spinal cord injury can also benefit from neuron axonal outgrowth produced by contact with hypoxanthine. As such, the present invention relates to methods of treatment of such an injury, the method comprising administering hypoxanthine to an individual in need thereof, to result in contact of the injured neuron with an effective amount of the hypoxanthine, to promote neuronal outgrowth.

Diseases, disorders, or conditions described herein as associated with injury to a neuron(s) can be treated in a subject by therapeutic administration of hypoxanthine. As such, aspects of the invention relate to methods of treating an individual for such diseases, disorders or conditions, described herein, by the methods herein described.

Diseases, disorders, or conditions associated with injury to a neuron(s) disorders or conditions in a subject, include, without limitation, damage to retinal ganglion cells; traumatic brain injury; stroke related injury; a cerebral aneurism related injury: a spinal cord injury, including monoplegia, diplegia, paraplegia, hemiplegia and quadriplegia; a neuroproliferative disorder or neuropathic pain syndrome.

Neuronal injury may also result from neurodegeneration which, for example, can be caused by or associated with neurotoxicity or a neurological disease, disorder or condition. Such disease or disorder includes, without limitation, Huntington's disease, Parkinson's disease, Multiple Sclerosis, Alzheimer's disease, multiple system atrophy (MSA), spino-cerebellar atrophy, motor neuronopathy, epilepsy or seizures, peripheral neuropathy, cerebral palsy, glaucoma (e.g., angle closure or open angle), age related loss of neurons or neuronal connectivity and related deterioration of sensory, motor, reflect, or cognitive abilities. Disease which are inflammatory and/or autoimmune disease can also cause or be indicated/associated with neuronal degeneration from which such neuronal injury can arise.

Injury to optic nerve neurons or retinal neurons may occur from the above listed causes. Injury to the optic nerve may further be the result of branch and central vein/artery occlusion, trauma, edema, angle-closure glaucoma, open-angle glaucoma. Retinal neurons may be injured by macular degeneration, age related macular degeneration, retinitis pigmentosa, retinal detachments, damage associated with laser therapy, and surgical light-induced iatrogenic retinopathy.

In some instances, damage to the neuron may be evidenced by loss of function or the presence of physical damage, e.g., visible nerve damage such as a break in an extension (axon or dendrite) or another form of lesion.

Subjects at risk for developing such neuronal damage can also be so treated by the methods described herein.

### Other Agents

The hypoxanthine can be contacted to the injured neuron(s) in combination with, or prior or subsequent to, other treatment regimes. One such treatment regimin is the use of anti-inflammatory agents such as methylprednisolone.

Hypoxanthine can further be contacted in combination with other axogenic factors, such as inosine, hexose or hexose derivatives, oncomodulin, nerve growth factor (NGF), brain-derived neurotrophic factor (BDNF), ciliary neurotrophic factor (CNTF). It can also be contacted/administered in combination with a cAMP activator.

The term “hexose derivative” refers to a hexose molecule that has one or more residues (e.g. esters, ethers, amino groups, amido groups, phosphate groups, sulphate groups, carboxyl groups, carboxy-alkyl groups, and combinations thereof) covalently or ionically attached to one or more of the molecules hydroxyl groups and able to produce a neurosalutary effect, as described in U.S. Patent Application 20050256059. Hexoses include mannose (D and L isomers), gulose, glucose-6-phosphate, aminomannose, mannose-6-phosphate (Phosporic acid mano-(3,4,5,6-tetrahydroxy-tetrahydro-pyran-2-ylmethy) ester).

While not wishing to be bound by a particular theory, it is believed that the cAMP and/or and axogenic factors may potentiate the activity of the hypoxanthine.

Preferably, the cAMP activator is non-hydrolyzable cAMP analogue, forskolin, adenylate cyclase activators, calcium ionophores, membrane depolarization, phosphodiesterase inhibitors, specific phosphodiesterase IV inhibitors, beta2-adrenoreceptor inhibitors or vasoactive intestinal peptide, and combinations thereof.

Administration of hypoxanthine to a subject, alone or in combinations described herein is to be made under conditions effective to stimulate nerve regeneration at the site of the injury and/or under conditions effective to at least partially restore nerve function through the injured spinal cord. Restoration of nerve function can be evidenced by restoration of nerve impulse conduction, a detectable increase in conduction action potentials, observation of anatomical continuity, restoration of more than one spinal root level, an increase in behavior or sensitivity, or a combination thereof. Administration is by a method which results in contacting the administered factors with the site of injury to thereby promote nerve regeneration (complete or partial).

In one embodiment, hypoxanthine is not co-administered or administered concurrently with xanthine oxidase, or any other oxidizing method to generate reactive O2.

### Pharmaceutically Acceptable Compositions

In one embodiment, the hypoxanthine which is administered in vivo is in a pharmaceutical composition or solution. The pharmaceutical composition or solution can further include one or more other exogenous agents (e.g., one or more axogenic factors, and/or cAMP activators) described herein as administered with or contacted in the presence of hypoxanthine. The pharmaceutical composition or solutions may optionally, be specifically formulated to exclude one or more such other agents. The pharmaceutical composition can optionally be formulated to exclude xanthine oxidase, or any other oxidizing method to generate reactive oxygen. In one embodiment, the pharmaceutical composition consists essentially of hypoxanthine and a pharmaceutically acceptable carrier. By the term “consists or consisting essentially of” is meant that the pharmaceutical composition does not contain any other active agents (e.g., modulators of neuronal growth such as, for example, NGF)).

In one embodiment, the pharmaceutical composition of the invention can be provided as a packaged formulation. The packaged formulation may include a pharmaceutical composition of the invention in a container and printed instructions for administration of the composition for treating a subject having a neuronal injury, and/or disease, disorder or condition associated with an injury neurons, as described herein.

Pharmaceutical compositions are considered pharmaceutically acceptable for administration to a living organism. For example, they are sterile, the appropriate pH, and ionic strength, for administration. They generally contain the hypoxanthine formulated in a composition within/in combination with a pharmaceutically acceptable carrier, also known in the art as excipients.

The pharmaceutically acceptable carrier is formulated such that it facilitates delivery of the active ingredient (e.g., hypoxanthine) to the target site. Such a carrier is suitable for administration and delivery to the target neuron. The pharmaceutically acceptable carrier will depend upon the location of the target neuron and the route of administration. For example, a typical carrier for intravenous administration of an agent is saline. The term “pharmaceutically acceptable carrier” includes, without limitation, any and all solvents, dispersion media, coatings, antibacterial and anti fungal agents, isotonic and absorption delaying agents, and the like that are physiologically compatible. For example, the carrier can be suitable for injection into the cerebrospinal fluid. The pharmaceutical composition can further be designed to provide protection of the hypoxanthine from unnecessary dispersion or degredation. The pharmaceutical composition may also contain additional ingredients such as stabilizers and disintegrants. Appropriate carriers and pharmaceutical compositions will be determined by the skilled practitioner.

In one embodiment, the pharmaceutical composition is easily suspended in aqueous vehicles and introduced through conventional hypodermic needles or using infusion pumps. Prior to introduction, the composition can be sterilized with, preferably, gamma radiation or electron beam sterilization, described in U.S. Pat. No. 436,742 the contents of which are incorporated herein by reference.

Additional examples of carriers are synthetic or natural polymers in the form of macromolecular complexes, nanocapsules, microspheres, or beads, and lipid-based formulations including oil-in-water emulsions, micelles, mixed micelles, synthetic membrane vesicles, and resealed erythrocytes.

In one embodiment, the pharmaceutically acceptable carrier comprises a polymeric matrix.

The terms “polymer” or “polymeric” are art-recognized and include a structural framework comprised of repeating monomer units which is capable of delivering a hypoxanthine or analog thereof such that treatment of a targeted condition, e.g., a CNS injury, occurs. The terms also include co-polymers and homopolymers e.g., synthetic or naturally occurring. Linear polymers, branched polymers, and cross-linked polymers are also meant to be included.

For example, polymeric materials suitable for forming the pharmaceutical composition employed in the present invention, include naturally derived polymers such as albumin, alginate, cellulose derivatives, collagen, fibrin, gelatin, and polysacchanides, as well as synthetic polymers such as polyesters (PLA, PLGA), polyethylene glycol, poloxomers, polyanhydrides, and pluronics. These polymers are biocompatible with the nervous system, including the central nervous system, they are biodegradable within the central nervous system without producing any toxic byproducts of degradation, and they possess the ability to modify the manner and duration of hypoxanthine release by manipulating the polymer's kinetic characteristics. As used herein, the term “biodegradable” means that the polymer will degrade over time by the action of enzymes, by hydrolytic action and/or by other similar mechanisms in the body of the subject. As used herein, the term “biocompatible” means that the polymer is compatible with a living tissue or a living organism by not being toxic or injurious and by not causing an immunological rejection.

Polymers can be prepared using methods known in the art (Sandler. S. R.; Karo, W. Polymer Syntheses; Harcourt Brace: Boston. 1994; Shalaby, W.; Ikada, Y.; Langer, R.: Williams, J. Polymers of Biological and Biomedical Significance (ACS Symposium Series 540; American Chemical Society: Washington, D.C. 1994). Polymers can be designed to be flexible; the distance between the bioactive side-chains and the length of a linker between the polymer backbone and the group can be controlled. Other suitable polymers and methods for their preparation are described in U.S. Pat. Nos. 5,455,044 and 5,576,018, the contents of which are incorporated herein by reference.

The polymeric formulations are preferably formed by dispersion of the hypoxanthine within liquefied polymer, as described in U.S. Pat. No. 4,883,666, the teachings of which are incorporated herein by reference or by such methods as bulk polymerization, interfacial polymerization, solution polymerization and ring polymerization as described in Odian G., Principles of Polymerization and ring opening polymerization, 2nd ed., John Wiley & Sons, New York, 1981, the contents of which are incorporated herein by reference. The properties and characteristics of the formulations are controlled by varying such parameters as the reaction temperature, concentrations of polymer and hypoxanthine, types of solvent used, and reaction times.

The hypoxanthine can be encapsulated in one or more pharmaceutically acceptable polymers, to form a microcapsule, microsphere, or microparticle, terms used herein interchangeably. Microcapsules, microspheres, and microparticles are conventionally free-flowing powders consisting of spherical particles of 2 millimeters or less in diameter, usually 500 microns or less in diameter. Particles less than 1 micron are conventionally referred to as nanocapsules, nanoparticles or nanospheres. For the most part, the difference between a microcapsule and a nanocapsule, a microsphere and a nanosphere, or microparticle and nanoparticle is size; generally there is little, if any, difference between the internal structure of the two. In one aspect of the present invention, the mean average diameter is less than about 45 μm, preferably less than 20 μm, and more preferably between about 0.1 and 10 μm.

In another embodiment, the pharmaceutical composition comprises lipid-based formulations. Any of the known lipid-based drug delivery systems can be used in the practice of the invention. For instance, multivesicular liposomes (MVL), multilamellar liposomes (also known as multilamellar vesicles or “MLV”). unilamellar liposomes, including small unilamellar liposomes (also known as unilamellar vesicles or “SUV”) and large unilamellar liposomes (also known as large unilamellar vesicles or “LUV”), can all be used so long as a sustained releaserate of the encapsulated hypoxanthine or analogue thereof can be established. In one embodiment, the lipid-based formulation can be a multivesicular liposome system. Methods of making controlled release multivesicular liposome drug delivery systems is described in PCT Application Serial Nos. U.S. 96/11642, U.S. 94/12957 and U.S. 94/04490, the contents of which are incorporated herein by reference. The composition of the synthetic membrane vesicle is usually a combination of phospholipids, usually in combination with steroids, especially cholesterol. Other phospholipids or other lipids may also be used.

Examples of lipids useful in synthetic membrane vesicle production include phosphatidylglycerols, phosphatidylcholines, phosphatidylserines, phosphatidylethanolamines, sphingolipids, cerebrosides, and gangliosides. Preferably phospholipids including egg phosphatidylcholine, dipalmitoylphosphatidylcholine, distearoylphosphatidylcholine, dioleoylphosphatidylcholine, dipalmitoylphosphatidylglycerol, and dioleoylphosphatidylglycerol are used.

In preparing lipid-based vesicles containing hypoxanthine, such variables as the efficiency of hypoxanthine encapsulation, lability of the hypoxanthine, homogeneity and size of the resulting population of vesicles, hypoxanthine-to-lipid ratio, permeability, instability of the preparation, and pharmaceutical acceptability of the formulation should be considered (see Szoka, et al., Annual Reviews of Biophysics and Bioengineering, 9:467, 1980; Deamer, et al., in Liposomes, Marcel Dekker, New York, 1983, 27; and Hope, et al., Chem. Phys. Lipids, 40:89, 1986, the contents of which are incorporated herein by reference).

In one, the pharmaceutical composition provides sustained delivery, e.g., “slow release” of the hypoxanthine to a subject for at least one, two, three, or four weeks after the pharmaceutical composition is administered to the subject.

As used herein, the term “sustained delivery” is intended to include continual delivery of a hypoxanthine or analogue thereof in vivo over a period of time following administration, preferably at least several days, a week or several weeks. Sustained delivery of the hypoxanthine can be demonstrated by, for example, the continued therapeutic effect of the hypoxanthine over time (e.g., by continued outgrowth of neurons over time). Alternatively, sustained delivery of the hypoxanthine may be demonstrated by detecting the presence of the hypoxanthine in vivo over time.

In one embodiment, the pharmaceutical composition provides sustained delivery of the hypoxanthine or analogue thereof to a subject for less than 30 days after the hypoxanthine or analogue thereof is administered to the subject. For example, the pharmaceutical composition, e.g., “slow release” formulation, can provide sustained delivery of the hypoxanthine to a subject for one, two, three or four weeks after the hypoxanthine is administered to the subject. Alternatively, the pharmaceutically composition may provide sustained delivery of the hypoxanthine to a subject for more than 30 days after the hypoxanthine is administered lo the subject.

### Administration

In one embodiment, administration of hypoxanthine to a subject, (e.g., in a pharmaceutical composition, with or without an additional axogenic factor described herein) results in the hypoxanthine directly contacting a neuron in need of regeneration. In another embodiment, the hypoxanthine and/or one or more of the factors do not directly contact the neuron, but contact the surrounding cells. Combinations of different forms of contacting with the various factors described herein are also envisioned.

In one embodiment, administration of a therapeutic amount of the compositions described herein is intended to produce a neurosalutary effect. The term “neurosalutary effect” means a response or result favorable to the health or function of a neuron, of a part of the nervous system, or of the nervous system generally. Examples of such effects include improvements in the ability of a neuron or portion of the nervous system to resist insult, to regenerate, to maintain desirable function, to grow or to survive. The phrase “producing a neurosalutary effect” includes producing or effecting such a response or improvement in function or resilience within a component of the nervous system. For example, examples of producing a neurosalutary effect would include stimulating axonal outgrowth after injury to a neuron; rendering a neuron resistant to apoptosis; rendering a neuron resistant to a toxic compound such as β-amyloid, ammonia, or other neurotoxins; reversing age-related neuronal atrophy or loss of function; or reversing age-related loss of cholinergic innervation.

Administration to the subject can be by any one or combination of a variety of methods (e.g., parenterally, enterally and/or topically). The appropriate method(s) will depend upon the circumstances of the individual (e.g. the location of the target neuron(s), the condition of the individual, the desired duration of the contact, whether local or systemic treatment is desired). The administration can be by any methods described herein that will result in contact of sufficient hypoxanthine to the target neuron to induce neuronal outgrowth. For instance, parenteral, enteral and topical administration can be used. The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. Enteral administration involves the esophagus, stomach, and small and large intestines (i.e., the gastrointestinal tract). The phrases “systemic administration,” “administered systemically”, “peripheral administration” and “administered peripherally” as used herein mean the administration of a compound other than directly into the central nervous system, such that it enters the animal's system and, thus, is subject to metabolism and other like processes, for example, subcutaneous administration. Administration may be topical (including ophthalmic, vaginal, rectal, intranasal, epidermal, and transdermal), oral or pulmonary administration, e.g., by inhalation or insufflation, or intracranial, e.g., intrathecal or intraventricular, administration, topically to the eye, or by intraocular injection.

Specific routes of administration and the dosage regimen will be determined by skilled clinicians, based on factors such as the exact nature of the condition being treated, the severity of the condition, and the age and general physical condition of the patient.

The invention also provides methods for stimulating the outgrowth of central nervous system neurons following an injury. The method involves administering to a subject hypoxanthine or analog thereof.

In one embodiment, contacting is achieved by administering the hypoxanthine to a subject by means to thereby contact the desired neuron(s). When administered following an injury, administration of hypoxanthine alone or in combinations described herein is to be made under conditions effective to stimulate nerve regeneration at the site of the injury and/or under conditions effective to at least partially restore nerve function through the injured nerve(s) (e.g. an injured spinal cord). Restoration of nerve function can be evidenced by restoration of nerve impulse conduction, a detectable increase in conduction action potentials, observation of anatomical continuity, restoration of more than one spinal root level, an increase in behavior or sensitivity, or a combination thereof. Under such circumstances, administration results in contacting the administered factors with the site of injury to thereby promote nerve regeneration (complete or partial).

The term “administering” to a subject includes dispensing, delivering or applying an active compound in a pharmaceutical formulation to a subject by any suitable route for delivery of the active compound to the desired location in the subject, (e.g., the injury, the injured neuron, or the site of desired outgrowth of the neuron). This includes, without limitation, delivery by either the parenteral or oral route, intramuscular injection, subcutaneous/intradermal injection, intravenous injection, buccal administration, transdermal delivery and administration by the rectal, colonic, vaginal, intranasal or respiratory tract route, intraocular, ocular. Another form of administration suitable for treatment of spinal cord injury is injection into the spinal column or spinal canal.

In one embodiment, the hypoxanthine or analog thereof is contacted in vivo by introduction into the central nervous system of a subject, e.g., into the cerebrospinal fluid of the subject. In certain aspects of the invention, the hypoxanthine or analog thereof is introduced intrathecally, e.g., into a cerebral ventricle, the lumbar area, or the cisterna magna. In another aspect, the hypoxanthine or analog thereof is introduced intraocullarly, to thereby contact retinal ganglion cells or the optic nerve. Modes of administration are described in U.S. Pat. No. 7,238,529.

In some circumstances, the methods described herein will not encompass direct administration into the brain of the subject (e.g., intrastriatal injection). In some circumstances, the methods described herein do not encompass intrathecal administration. That is to say, contacting of the neuron (e.g. the target neuron) is not accomplished by intrathecal administration and/or by direct administration into the brain of the subject (e.g., intrastriatal injection).

In one embodiment, administration occurs following neuronal injury in the subject, not prior to or at the time of neuronal injury.

In another embodiment of the invention, the hypoxanthine formulation is administered into a subject intrathecally. As used herein, the term “intrathecal administration” is intended to include delivering a hypoxanthine formulation directly into the cerebrospinal fluid of a subject, by techniques including lateral cerebroventricular injection through a burrhole or cisternal or lumbar puncture or the like (described in Lazorthes et al. Advances in Drug Delivery Systems and Applications in Neurosurgery, 143-192 and Omaya et al., Cancer Drug Delivery, 1: 169-179, the contents of which are incorporated herein by reference). The term “lumbar region” is intended to include the area between the third and fourth lumbar (lower back) vertebrae. The term “cisterna magna” is intended to include the area where the skull ends and the spinal cord begins at the back of the head. The term “cerebral ventricle” is intended to include the cavities in the brain that are continuous with the central canal of the spinal cord. Administration of a hypoxanthine to any of the above mentioned sites can be achieved by direct injection of the hypoxanthine formulation or by the use of infusion pumps. For injection, the hypoxanthine formulation of the invention can be formulated in liquid solutions, preferably in physiologically compatible buffers such as Hank's solution or Ringer's solution. In addition, the hypoxanthine formulation may be formulated in solid form and re-dissolved or suspended immediately prior to use. Lyophilized forms are also included. The injection can be, for example, in the form of a bolus injection or continuous infusion (e.g., using infusion pumps) of the hypoxanthine formulation.

In one embodiment of the invention, said hypoxanthine formulation is administered by lateral cerebro ventricular injection into the brain of a subject in the inclusive period from the time of the injury to 100 hours thereafter. The injection can be made, for example, through a burr hole made in the subject's skull. In another embodiment, said encapsulated therapeutic agent is administered through a surgically inserted shunt into the cerebral ventricle of a subject in the inclusive period from the time of the injury to 100 hours thereafter. For example, the injection can be made into the lateral ventricles, which are larger, even though injection into the third and fourth smaller ventricles can also be made.

In yet another embodiment, said hypoxanthine formulation is administered by injection into the cisterna magna, or lumbar area of a subject in the inclusive period from the time of the injury to 100 hours thereafter.

### Duration and Levels of Administration

Contacting of the injured neuron(s) can be anytime following the injury. In one embodiment, the injured neuron is contacted within 96 hours of formation of the lesion on the neuron to be contacted, and more preferably within 72, 48, 24, or 12 hours.

The treatment of a subject may likewise begin anytime following the injury. In one embodiment, the treatment progresses upon detection or suspicion of the injury. For example, the treatment can be 6 hr, 12, hr, 18 hr, or 24 hours post injury. Benefit is also expected to be had from treatment that takes place considerably longer after the injury. The injury may have occurred more than three months prior to the treatment, more than one month prior, more than three weeks prior to the treatment, or more than two weeks prior to the treatment, more than one week prior to the treatment or from between 1-6 days prior to the treatment.

In one embodiment, the hypoxanthine (e.g., in the form of a pharmaceutical composition) described herein is contacted to a neuron, and/or administered to the subject in the period from the time of injury to 100 hours, for example within 24, 12 or 6 hours after the injury has occurred.

The pharmaceutical composition, used in the method of the invention, contains a therapeutically effective amount of the hypoxanthine. A “therapeutically effective amount” refers to an amount effective, at dosages and for periods of time necessary, to achieve the desired result (e.g., a neurosalutary effect, detectable neuronal outgrowth). A therapeutically effective amount of the hypoxanthine may vary according to factors such as the disease state, age, and weight of the subject, and the ability of the hypoxanthine or analogue thereof (alone or in combination with one or more other agents) to elicit a desired response in the subject. Dosage regimens may be adjusted to provide the optimum therapeutic response. A therapeutically effective amount is also one in which any toxic or detrimental effects of the hypoxanthine or analogue thereof are outweighed by the therapeutically beneficial effects.

The term “therapeutically effective amount” refers to an amount that is sufficient to effect a therapeutically reduction in a symptom associated with neuronal injury, disease, disorder or condition described herein, when administered to a typical subject who has said injury, disease, disorder, condition. A therapeutically significant reduction in a symptom is, e.g. about 10%, about 20%, about 30%, about 40%, about 50%, about 60%, about 70%, about 80%, about 90%, about 100%, about 125%, about 150% or more as compared to a control or non-treated subject. In some embodiments the term “therapeutically effective amount” refers to the amount that is safe and sufficient to prevent or delay the development and further spread of neuronal injury, disease, or other disease symptoms. The amount can also cure or cause the disease, disorder or condition to go into remission, slow the course of, or otherwise inhibit progression.

A non-limiting range for a therapeutically effective concentration of hypoxanthine is 5 μM to 1 mM. A non-limiting range for a therapeutically effective concentration of hypoxanthine is at least 25 μM to 1 mM. In a particularly preferred embodiment, the therapeutically effective concentration of the hypoxanthine is 10-25 μM, or 25-50 μM. In a particularly preferred embodiment, the therapeutically effective concentration of the hypoxanthine is 25-50 μM, 50-100 μM, or 100-150 μM. It is to be noted that dosage values may vary with the severity of the condition to be alleviated. It is to be further understood that for any particular subject, specific dosage regimens should be adjusted over time according to the individual need and the professional judgment of the person administering or supervising the administration of the hypoxanthine or analogue thereof and that dosage ranges set forth herein are exemplary only and are not intended to limit the scope or practice of the claimed invention.

A therapeutically effective amount or dosage of a the hypoxanthine ranges from about 0.001 to 30 mg/kg body weight, with other ranges of the invention including about 0.01 to 25 mg/kg body weight, about 0.1 to 20 mg/kg body weight, about 1 to 10 mg/kg, 2 to 9 mg/kg, 3 to 8 mg/kg, 4 to 7 mg/kg, and 5 to 6 mg/kg body weight. A non-limiting range for a therapeutically effective in vivo concentration in tissue containing the injury is 5 μM to 5 mM.

In one embodiment, administration occurs following neuronal injury in the subject, not prior to or at the time of neuronal injury.

### In Vitro Treatment of Neurons

Neurons can further be contacted with a therapeutically effective amount of a hypoxanthine, in vitro or ex vivo. Accordingly, neuron cells can be isolated from a subject and grown in vitro, using techniques well known in the art. Briefly, a neuron cell culture can be obtained by allowing neuron cells to migrate out of fragments of neural tissue adhering to a suitable substrate (e.g., a culture dish) or by disaggregating the tissue, e.g., mechanically or enzymatically, to produce a suspension of neuron cells. For example, the enzymes trypsin, collagenase, elastase, hyaluronidase, DNase, pronase, dispase, or various combinations thereof can be used. Trypsin and pronase give the most complete disaggregation but may damage the cells. Collagenase and dispase give a less complete dissagregation but are less harmful. Methods for isolating tissue (e.g., neural tissue) and the disaggregation of tissue to obtain cells (e.g., CNS neuron cells) are described in Freshney R. I., Culture of Animal Cells,

A Manual of Basic Technique, Third Edition, 1994, the contents of which are incorporated herein by reference.

Such cells can be subsequently contacted with hypoxanthine at levels and for a duration of time as described above. Once modulation of neuronal outgrowth has been achieved in the neuron cells, these cells can be re-administered to the subject, e.g., by implantation.

### Hypoxanthine Eluting Devices

The invention also provides hypoxanthine-eluting or hypoxanthine-impregnated implantable solid or semi-solid devices, for implantation into the CNS or the PNS. Examples of CNS implantable devices include polymeric microspheres (e.g. see Benny et al., Clin Cancer Res. (2005) 11:768-76) or wafers (e.g. see Tan et al., J Pharm Sci. (2003) 4:773-89), biosynthetic implants used in tissue regeneration after spinal cord injury (reviewed by Novikova et al., Curr Opin Neurol. (2003) 6:711-5), biodegradable matrices (see e.g. Dumens et al., Neuroscience (2004) 125:591-604), biodegradable fibers (see e.g. U.S. Pat. No. 6,596,296), osmotic pumps, stents, adsorbable gelatins (see e.g. Doudet et al., Exp Neurol. (2004) 189:361-8). Preferred devices are particularly tailored, adapted, designed or designated for CNS implantation. The implantable device may contain one or more additional agents used to promote or facilitate neural regeneration, as described herein.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means ±5%.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified herein are expressly incorporated herein by reference in their entirety. This incorporation is, for example, for the purpose of describing and disclosing the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention may be as defined in any one of the following numbered paragraphs.

1. A method of promoting neuronal outgrowth in a neuron comprising contacting the neuron with an effective amount of hypoxanthine, to thereby promote neuronal outgrowth of the neuron.

2. The method of paragraph 1, wherein hypoxanthine is contacted in the absence of xanthine oxidase and/or in the absence of exogenous nerve growth factor (NGF), and/or in the absence of exogenous D-mannose, and/or in the absence of exogenous oncomodulin, and/or in the absence of exogenous TGF-B.

3. The method of paragraphs 1 and 2, wherein the neuron is an optic nerve neuron or a retinal neuron.

4. The method of paragraphs 1-3, wherein the contacting occurs in vivo.

5. The method of paragraphs 1-3, wherein the contacting occurs in vitro.

6. The method of paragraphs 1-5, wherein the neuron is an injured neuron.

7. The method of paragraphs 1-6, wherein the neuron is selected from the group consisting of central nervous system (CNS) neuron and peripheral nervous system (PNS) neuron.

8. The method of paragraphs 1-7, wherein the neuron is an optic nerve neuron or a retinal neuron.

9. The method of paragraph 8, wherein the optic nerve neuron is an injured neuron, and injury to the optic nerve neuron is the result of branch and central vein/artery occlusion, trauma, edema, angle-closure glaucoma, open-angle glaucoma.

10. The method of paragraph 8 , wherein the retinal neuron is injured as the result of macular degeneration, age related macular degeneration, retinitis pigmentosa, retinal detachments, damage associated with laser therapy, and surgical light-induced iatrogenic retinopathy.

11. The method of paragraphs 1-4, 6-10, wherein contacting occurs in vivo, by administration to a subject, to thereby contact the neuron.

12. The method of paragraph 11, wherein the subject suffers from neurological injury that results from a disease or condition of the subject.

13. The method of paragraph 12, wherein the disease or condition is stroke, spinal cord injury, Huntington's disease, Parkinson's disease, Alzheimer's disease, multiple system atrophy (MSA), spino-cerebellar atrophy, motor neuropathy, epilepsy or seizures, peripheral neuropthy, cerebral palsy, glaucoma, age related loss of neurons or neuronal connectivity and related deterioration of sensory, motor, reflect, or cognitive abilities.

14. The method of paragraph 11 wherein the subject suffers from an injury caused by or associated with peripheral neuropathies and/or peripheral nerve damage associated with spinal cord injury.

15. The method of paragraph 14, wherein the peripheral neuropathy is diabetic neuropathy, virus-associated neuropathy, botulism-related neuropathy; toxic polyneuropathy, nutritional neuropathy, angiopathic neuropathy, sarcoid-associated neuropathy; carcinomatous neuropathy; compression neuropathy, and/or hereditary neuropathy.

16. The method of paragraph 11, wherein the subject suffers from a neurological injury resulting from a trauma.

17. The method of paragraph 11, wherein the subject suffers from a neurological injury resulting from exposure to a toxin.

18. The method of paragraphs 11-17, wherein administration is parenteral, enteral and/or topical.

19. The method of paragraphs 11-17, wherein contacting is achieved by ocular administration.

20. The method of paragraphs 1-19, further comprising contacting the neuron with

D-mannose.

21. The method of paragraphs 1-20, further comprising contacting the neuron with a cAMP activator.

22. The method of paragraph 21, wherein the cAMP activator is non-hydrolyzable cAMP analogues, adenylate cyclase activators, calcium ionophores, phosphodiesterase inhibitors, specific phosphodiesterase IV inhibitors, beta2-adrenoreceptor inhibitors or vasoactive intestinal peptide.

23. The method of paragraphs 1-22, further comprising contacting the neuron with oncomodulin.

24. The method of paragraphs 1-23, further comprising contacting the neuron with TGF-β.

25. A method of treating a disorder or condition in a subject associated with injury to a neuron(s) comprising, administering a therapeutically effective amount of hypoxanthine to the subject sufficient to promote neuronal outgrowth in the subject.

26. The method of paragraph 25, wherein the disorder or condition is acute spinal cord damage, stroke, Huntington's disease, Parkinson's disease, Multiple Sclerosis, Alzheimer's disease, multiple system atrophy (MSA), spino-cerebellar atrophy, motor neuronopathy, epilepsy or seizures, peripheral neuropthy, cerebral palsy, glaucoma, macular degeneration, age related macular degeneration, retinitis pigmentosa, retinal detachments, damage associated with laser therapy (including photodynamic therapy), and surgical light-induced iatrogenic retinopathy.

27. A method of treating a neuronal injury in a subject comprising, administering a therapeutically effective amount of hypoxanthine to the subject sufficient to promote neuronal outgrowth in the subject.

28. The method of paragraphs 25-27 wherein the hypoxanthine is administered parenterally, enterally and/or topically.

29. The method of paragraphs 25-28, further comprising administering an effective amount of D-mannose to the subject.

30. The method of paragraphs 25-29, further comprising administering an effective amount of a cAMP activator to the subject.

31. The method of paragraphs 25-30, further comprising administering an effective amount of oncomodulin to the subject.

32. The method of paragraphs 25-31, further comprising administering an effective amount of TGF-β to the subject.

33. The method of paragraphs 25-28, wherein the hypoxanthine is administered in the absence of xanthine oxidase and/or in the absence of exogenous nerve growth factor (NGF), and/or in the absence of exogenous D-mannose, and/or in the absence of exogenous oncomodulin, and/or in the absence of exogenous TGF-B.

34. A pharmaceutical composition comprising hypoxanthine and a pharmaceutically acceptable carrier.

35. The pharmaceutical composition of paragraph 34, further comprising a cAMP modulator, and a pharmaceutically acceptable carrier.

36. The pharmaceutical composition of paragraph 35 wherein the cAMP modulator is non-hydrolyzable cAMP analogues, forskolin, adenylate cyclase activators, macrophage-derived factors that stimulate cAMP, macrophage activators, calcium ionophores, phosphodiesterase inhibitors, specific phosphodiesterase IV inhibitors, beta2-adrenoreceptor inhibitors or vasoactive intestinal peptide.

37. The pharmaceutical composition of paragraphs 34 or 35 further comprising one or more additional axogenic factors.

38. The pharmaceutical composition of paragraph 37 wherein the one or more other axogenic factors is mannose, a mannose derivative, inosine, oncomodulin, and combinations thereof.

The invention is further illustrated by the following examples, which should not be construed as further limiting.

### EXAMPLES

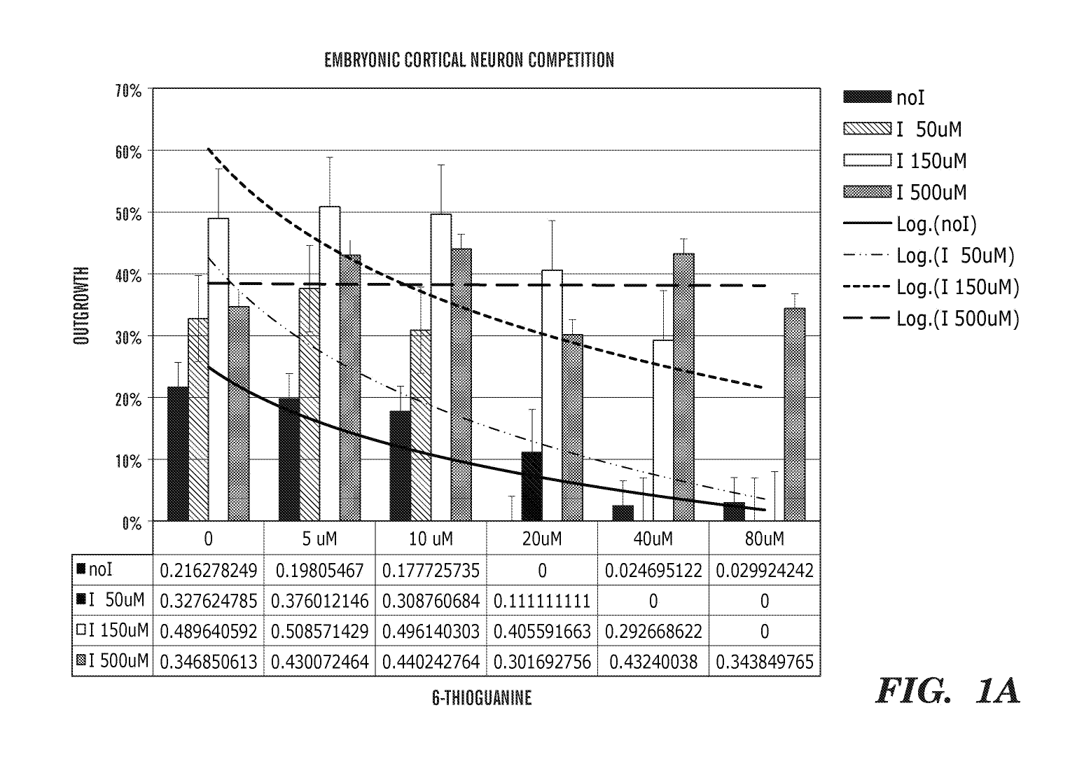

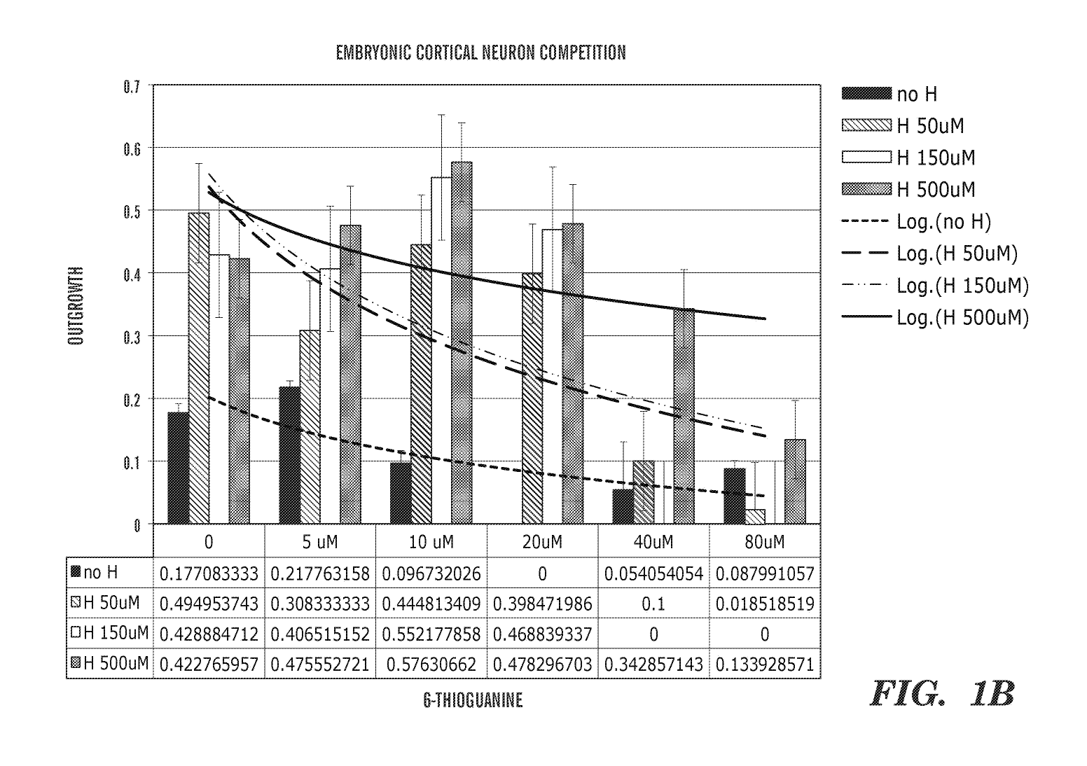

These experiments were done on embryonic cortical neurons. The forebrains of postnatal day 15 rat pups were dissected and plated in media containing serum. After 3 hours the medium was exchanged for Medium E (Irwin et al. 2006) (Proc Nati Acad Sci U S A. 2006 Nov 28;103(48):18320-5). The cells were treated the following day with inosine or hypoxanthine, in the presence and absence of the indicated amount of the inhibitor 6-thioguanine (6-TG), and allowed to grow for 3 days. The 6-TG was added 1 hour before the hypoxanthine or inosine. Following the three days of growth, the cells were fixed and stained with Taul antibody and a fluorescent secondary antibody. Axons were then counted. 6-TG competitively inhibited the actions of both inosine and hypoxanthine. FIG. 1 presents the data obtained. FIG. 1 shows similar dose response curves are obtained with inosine and hypoxanthine, and that each is similarly inhibited by 6-TG (FIG. 1A and 1B (charts 8 and 9)). This indicates that inosine and hypoxanthine behave similarly with respect to induction of axon outgrowth in neurons.

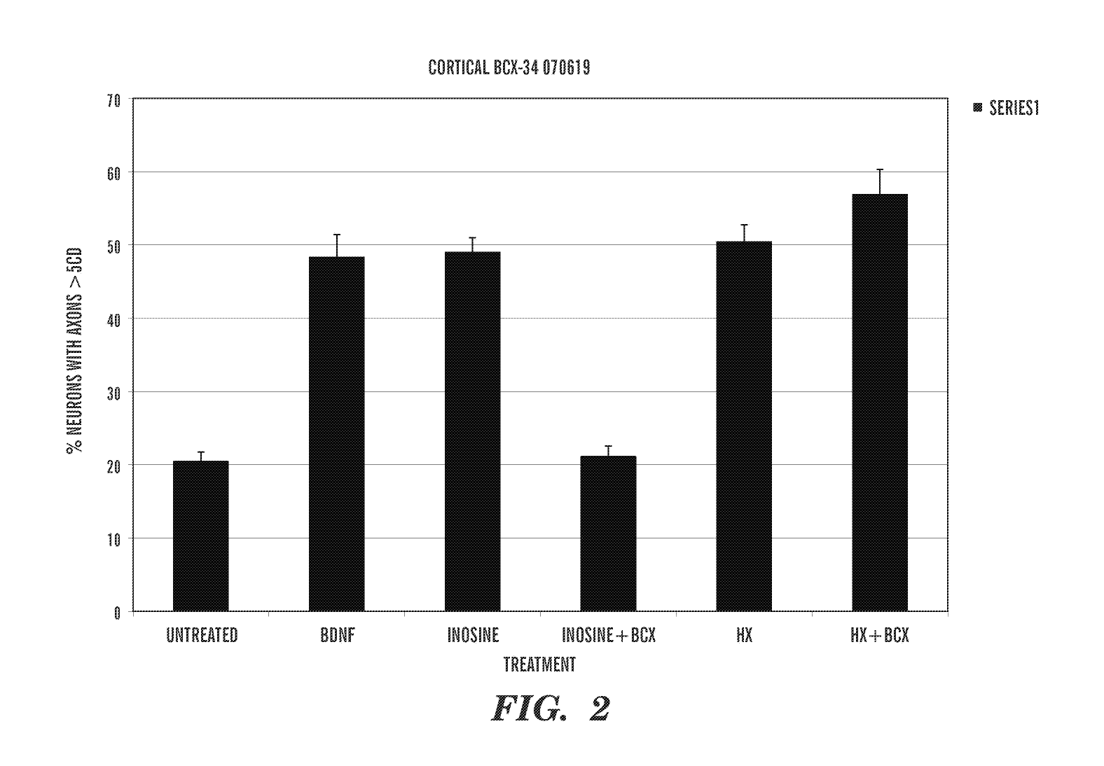

In a similar experiment, embryonic cortical neurons were given brain derived neurotrophic factor (BDNF) (2 ng/ml), inosine, or hypoxanthine (100 μM). Inosine and hypoxanthine were given in the presence or absence of the inhibitor BCX-34 (BCX). BCX-34 blocks the conversion of inosine to hypoxanthine. BCX-34 (50 μM) was added 1 hour before the hypoxanthine or inosine. The cells were then scored for neuron axonal outgrowth. The data collected is represented in FIG. 2. Both inosine and hypoxanthine induced axonal outgrowth similarly to BDNF. The BCX-34, known to block the conversion of inosine to hypoxanthine, blocked axon outgrowth of cells exposed to inosine, but did not negatively impact the growth of cells exposed to hypoxanthine. These results indicate that inosine must be converted to hypoxanthine in order to stimulate outgrowth.

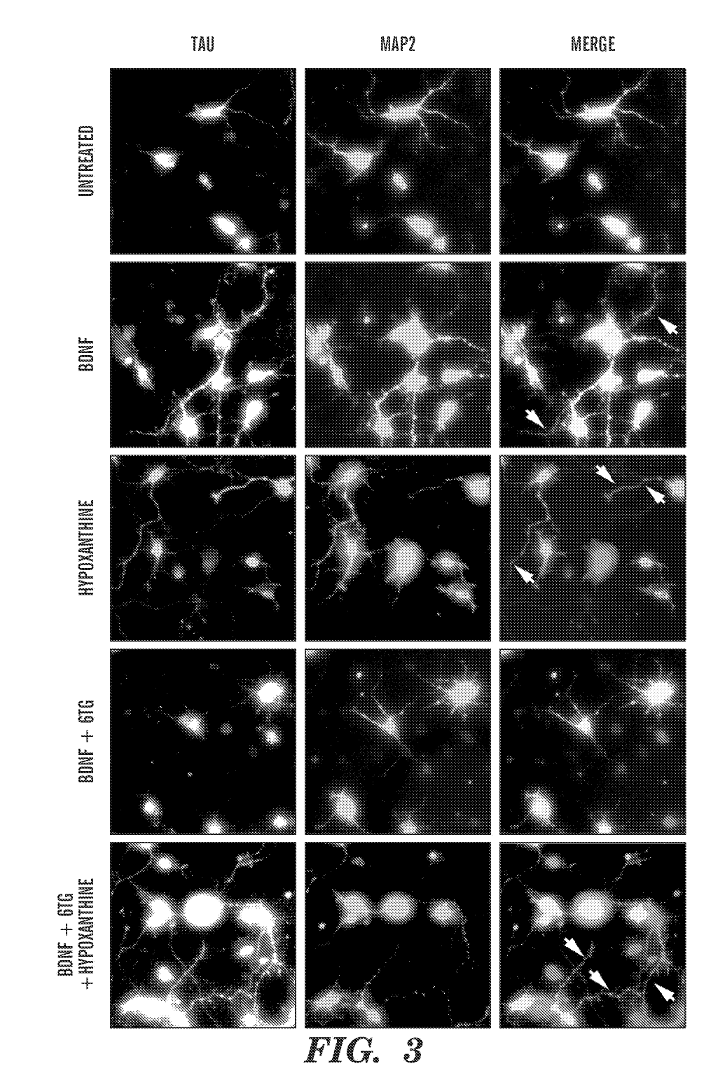

Embryonic cortical neurons were grown in the presence of BDNF (2 ng/ml), hypoxanthine (100 μM), BDNF (2 ng/ml)+6-thioguanine (20 μM) , or BDNF (2 ng/ml)+6-thioguanine (20 μM)+hypoxanthine (100 μM), as described above. The cells were then fixed and stained with either antibodies to Taul, antibodies to map2, or both antibodies to Taul and antibodies to map2, and corresponding fluorescent secondary antibodies, in order to visualize axonal outgrowth. Representative photographs of the stained cells are shown in FIG. 3. Treatment of the cells with BDNF or hypoxanthine caused axonal outgrowth. The BDNF stimulated outgrowth was inhibited by 6-TG and this inhibition was overcome by adding high amounts of hypoxanthine. It has also been shown that hypoxanthine activates Mst3b (data not shown).

1. A method of promoting neuronal outgrowth in a neuron comprising contacting the neuron with an effective amount of hypoxanthine, to thereby promote neuronal outgrowth of the neuron.

2. The method of claim 1, wherein hypoxanthine is contacted in the absence of one or more agents selected from the group consisting of exogenous xanthine oxidase, exogenous nerve growth factor (NGF), exogenous D-mannose, exogenous oncomodulin, and exogenous TGF-B.

3. The method of claim 1, wherein the neuron is an optic nerve neuron or a retinal neuron.

4. The method of claim 1, wherein the contacting occurs in vivo.

5. The method of claim 1, wherein the contacting occurs in vitro.

6. The method of claim 1, wherein the neuron is an injured neuron.

7. The method of claim 1, wherein the neuron is selected from the group consisting of central nervous system (CNS) neuron and peripheral nervous system (PNS) neuron.

8.-15. (canceled)

16. The method of claim 27, wherein the neuronal injury results from a trauma or exposure to a toxin.

17. (canceled)

18. The method of claim 4, wherein contacting occurs by parenteral, enteral or topical administration to a subject.

19. (canceled)

20. The method of claim 1, further comprising contacting the neuron with an axogenic factor selected from the group consisting of oncomodulin, TGF-β, and D-mannose.

21. The method of claim 1, further comprising contacting the neuron with a cAMP activator.

22.-25. (canceled)

26. The method of claim 27, wherein the neuronal injury is associated with a disease or condition in the subject selected from the group consisting of acute spinal cord damage, stroke, Huntington's disease, Parkinson's disease, Multiple Sclerosis, Alzheimer's disease, multiple system atrophy (MSA), spino-cerebellar atrophy, motor neuronopathy, epilepsy or seizures, peripheral neuropthy, cerebral palsy, glaucoma, macular degeneration, age related macular degeneration, retinitis pigmentosa, retinal detachments, damage associated with laser therapy, and surgical light-induced iatrogenic retinopathy.

27. A method of treating a neuronal injury in a subject comprising, administering a therapeutically effective amount of hypoxanthine to the subject sufficient to promote neuronal outgrowth in the subject.

28. The method of claim 27 wherein the hypoxanthine is administered parenterally, enterally or topically.

29. (canceled)

30. The method of claim 27, further comprising administering an effective amount of a cAMP activator to the subject.

31. The method of claim 27, further comprising administering an effective amount of an axogenic factor selected from the group consisting of oncomodulin, D-mannose and TGF-β to the subject.

32. (canceled)

33. The method of claim 27, wherein the hypoxanthine is administered in the absence of one or more agents selected from the group consisting of exogenous xanthine oxidase, exogenous nerve growth factor (NGF), exogenous D-mannose, exogenous oncomodulin, and exogenous TGF-β.

34. A pharmaceutical composition comprising hypoxanthine and a pharmaceutically acceptable carrier.

35. The pharmaceutical composition of claim 34, further comprising a cAMP modulator.

36. (canceled)

37. The pharmaceutical composition of claim 34, further comprising one or more additional axogenic factors selected from the group consisting of D-mannose, oncomodulin, and inosine.

38. (canceled)
